(19) United States
(12) Patent Application Publication
(10) Pub. No.: US 2004/0147830 A1
(43) Pub. Date: Jul. 29, 2004

(54) Method and system for use of biomarkers in diagnostic imaging

(75) Inventors: Kevin J. Parker, Rochester, NY (US); Jose Tamez-Pena, Rochester, NY (US); Saara Marjatta Sofia Totterman, Rochester, NY (US); Edward Ashton, Webster, NY (US)

Correspondence Address: BLANK ROME LLP, 600 NEW HAMPSHIRE AVENUE, N.W., WASHINGTON, DC, 20037, US

(73) Assignee: VirtualScopics

(21) Appl. No.: 10/352,867

(22) Filed: Jan. 29, 2003

(51) Int. Cl. A61B 5/00; G06K 9/00; A61B 5/05

(52) U.S. Cl. 600/407; 382/128; 600/426; 128/920; 128/922

(57) ABSTRACT

In a human or animal organ or other region of interest, specific objects, such as liver metastases and brain lesions, serve as indicators, or biomarkers, of disease. In a three-dimensional image of the organ, the biomarkers are identified and quantified. Multiple three-dimensional images can be taken over time, in which the biomarkers can be tracked over time. Statistical segmentation techniques are used to identify the biomarker in a first image and to carry the identification over to the remaining images. Regions of normal and abnormal parameters within the 3D biomarker structure are identified. The information is used to highlight or visualize abnormal regions on the original 2D tomographic images.

### FIELD OF THE INVENTION

The present invention is directed to the assessment of certain biologically or medically significant characteristics of bodily structures, known as biomarkers, and more particularly to the use of biomarkers in diagnostic imaging. Measurements of biomarkers, and identification of abnormal biomarker parameters, are used to create Computer Assisted Localization (CAL) which integrates the traditional image information utilized by radiologists, with advanced 3D and 4D quantitative information from biomarkers.

### DESCRIPTION OF RELATED ART

The measurement of internal organs and structures from CT, MRI, ultrasound, PET, and other imaging data sets is an important objective in many fields of medicine. For example, in obstetrics, the measurement of the biparietal diameter of the fetal head gives an objective indicator of fetal growth. Another example is the measurement of the hippocampus in patients with epilepsy to determine asymmetry (Ashton E. A., Parker K. J., Berg M. J., and Chen C. W. “A Novel Volumetric Feature Extraction Technique with Applications to MR Images,” IEEE Transactions on Medical Imaging 16:4, 1997). The measurement of the thickness of the cartilage of bone is another research area (Stammberger, T., Eckstein, F., Englmeier, K-H., Reiser, M. “Determination of 3D Cartilage Thickness Data from MR Imaging: Computational Method and Reproducibility in the Living,” Magnetic Resonance in Medicine 41, 1999; and Stammberger, T., Hohe, J., Englmeier, K-H., Reiser, M., Eckstein, F. “Elastic Registration of 3D Cartilage Surfaces from MR Image Data for Detecting Local Changes in Cartilage Thickness,” Magnetic Resonance in Medicine 44, 2000). Those measurements are quantitative assessments that, when used, are typically based on manual intervention by a trained technician or radiologist. For example, trackball or mouse user interfaces are commonly used to derive measurements such as the biparietal diameter. User-assisted interfaces are also employed to initiate some semi-automated algorithms (Ashton et al). The need for intensive and expert manual intervention is a disadvantage, since the demarcations can be tedious and prone to a high inter- and intra-observer variability. Furthermore, the typical application of manual measurements within 2D slices, or even sequential 2D slices within a 3D data-set, is not optimal, since tortuous structures, curved structures, and thin structures are not well characterized within a single 2D slice, leading again to operator confusion and high variability in results.

The need for accurate and precise measurements of organs, tissues, structures, and sub-structures continues to increase. For example, in following the response of a disease to a new therapy, the accurate representation of 3D structures is vital in broad areas such as neurology, oncology, orthopedics, and urology. Another important need is to track those measurements of structures over time, to determine if, for example, a tumor is shrinking or growing, or if the thin cartilage is further deteriorating. If the structures of interest are tortuous, or thin, or curved, or have complicated 3D shapes, then the manual determination of the structure from 2D slices is tedious and prone to errors. If those measurements are repeated over time on successive scans, then inaccurate trend information can unfortunately be obtained. For example, subtle tumor growth along an out-of-plane direction can be lost within poor accuracy and precision and high variability from manual or semi-manual measurements.

Yet another problem with conventional methods is that they lack sophistication and are based on “first order” measurements of diameter, length, or thickness. With some semi-manual tracings, the measurement is extended to a two-dimensional area or a three-dimensional volume (Ashton et al). Those traditional measurements can be insensitive to small but important changes. For example, consider the case of a thin structure such as the cartilage. Conventional measurements of volume and thickness will be insensitive to the presence or absence of small pits in the cartilage, yet those defects could be an important indicator of a disease process.

The prior art is capable of assessing gross abnormalities or gross changes over time. However, the conventional measurements are not well suited to assessing and quantifying subtle abnormalities, or subtle changes, and are incapable of describing complex topology or shape in an accurate manner. Furthermore, manual and semi-manual measurements from raw images suffer from a high inter-space and intra-observer variability. Also, manual and semi-manual measurements tend to produce ragged and irregular boundaries in 3D, when the tracings are based on a sequence of 2D images.

### SUMMARY OF THE INVENTION

In light of the aforementioned disadvantages, it becomes apparent that there is a clear need for improved imaging systems and methods. Moreover, there is a need for an invention which utilizes “higher order” measurements to provide a previously unknown degree of resolution and quantification of biomarkers from their respective medical imaging data sets. Additionally, there is a need for an invention that incorporates these highly accurate and definitive images into a contiguous temporal framework, thus providing an accurate definition of trends over time.

Clearly, a need exists for improvement upon: (1) earlier methods of assessing and quantifying structures; (2) localizing regions of abnormal biomarker parameters; (3) tracking change(s) of biological structure(s) and/or sub-structures over time; and (4) incorporating “higher order” measurements. More precisely, there is a clear need for measurements that are more accurate and precise, with lower variability than conventional manual or semi-manual methods. There is furthermore a need for measurements that are accurate over time, as repeated measurements are made. There is furthermore a need for measurements based on high-resolution data sets, such that small defects, tortuous objects, thin objects, and curved objects, can be quantified. Furthermore there is a need for measurements, parameters, and descriptors which are more sophisticated, more representative and more sensitive to subtle changes than the simple “first order” measurements of length, diameter, thickness, area and volume.

Finally, there is a need to juxtapose or combine the information that is available from measurements of biomarkers of a patient, with the conventional image information that is traditionally reviewed by the radiologist. For example, the examination of a single MRI or CT image of a complicated 3D structure such as the hippocampus of the brain or the meniscus of the knee, is insufficient to assess many subtle changes or subtle abnormalities, or parameter values that are outside those expected in normals in the structure, even though these subtle changes can be assessed quantitatively by 3D segmentation and quantitative measurement. The results of the 3D measurements, and particularly any abnormalities that are found, are needed to localize regions of interest in the original imaging scan planes that should be examined closely by the radiologist or surgeon. What is needed is a method and system for Computer Assisted Localization (CAL) which superimposes or combines information from the 3D biomarker measurements with the conventional scan plane image.

To achieve the above and other objects, the present invention is directed to a system and method for accurately and precisely identifying important structures and sub-structures, their normalities and abnormalities, and their specific topological and morphological characteristics—all of which are sensitive indicators of disease processes and related pathology. Then, the biomarker information, particularly local regions of abnormal biomarker parameters, is superimposed or integrated back onto the original scan plane image information. In this way the conventional imaging information, rich in texture and 2D anatomical details, can be combined with 3D biomarker information that can be used to localize areas in the 2D image that should be examined more closely by the radiologist, surgeon, or evaluator. This combination of information and localization of regions of interest is called Computer Assisted Localization (CAL). Note that CAL is different from the approach of Computer Assisted Diagnosis (CAD), in that the general focus of CAD is the detection and classification of specific diseases such as breast cancer based on pattern recognition.

The preferred technique is to identify the biomarkers based on automatic techniques that employ statistical reasoning to segment the biomarker of interest from the surrounding tissues (the statistical reasoning is given in Parker et al., U.S. Pat. No. 6,169,817, whose disclosure is hereby incorporated by reference in its entirety into the present disclosure). This can be accomplished by fusion of a high resolution scan in the orthogonal, or out-of-plane direction, to create a high resolution voxel data set (Peña, J.-T., Totterman, S. M. S., Parker, K. J. “MRI Isotropic Resolution Reconstruction from Two Orthogonal Scans,” SPIE Medical Imaging, 2001). In addition to the assessment of subtle defects in structures, this high-resolution voxel data set enables more accurate measurement of structures that are thin, curved, or tortuous. More specifically, this invention improves the situation in such medical fields as oncology, neurology, and orthopedics. In the field of oncology, for example, the invention is capable of identifying tumor margins, specific sub-components such as necrotic core, viable perimeter, and development of tumor vasculature (angiogenesis), which are sensitive indicators of disease progress or response to therapy. Similarly, in the fields of neurology and orthopedics, the invention is capable of identifying characteristics of both the whole brain and prosthesis wear, respectively.

Generally speaking, biomarkers are biological structures and are thus subject to change in response to a variety of things. For example, the brain volume in a patient with multiple sclerosis may diminish after a period of time. In this case, a disease (multiple sclerosis) has caused a change in a biomarker (brain volume). More information on biomarkers and their use is found in the applicants' co-pending U.S. patent application Ser. No. 10/189,476, filed Jul. 8, 2002, whose disclosure is hereby incorporated by reference in its entirety into the present disclosure. For a physician attempting to effectively monitor the progress of a disease via an image-based platform, an accurate, precise and temporally contiguous picture of the progress of the disease is needed. In light of the current state of imaging technology, however, the ability to accurately and precisely monitor disease progress on an image-based platform is non-existent.

It is desirable to accurately and precisely monitor the trends in biomarkers over time. For example, it is useful to monitor the condition of trabecular bone in patients with osteoarthritis. The inventors have discovered that extracting a biomarker using statistical tests and treating the biomarker over time as a four-dimensional (4D) object, with an automatic passing of boundaries from one time interval to the next, can provide a highly accurate and reproducible segmentation from which trends over time can be detected. This preferred approach is defined in the above-cited U.S. Pat. No. 6,169,817. Thus, this invention improves the situation by combining selected biomarkers that themselves capture subtle pathologies, with a 4D approach to increase accuracy and reliability over time, to create a sensitivity that has not been previously obtainable.

Another feature which may be used in the present invention is that of “higher order” measures. Although the conventional measures of length, diameter, and their extensions to area and volume are useful quantities, they are limited in their ability to assess subtle but potentially important features of tissue structures or substructures. The example of the cartilage was already mentioned, where measures of gross thickness or volume would be insensitive to the presence or absence of small defects. Thus, the present invention preferably uses “higher order” measures of structure and shape to characterize biomarkers. “Higher order” measures are defined as any measurements that cannot be extracted directly from the data using traditional manual or semi-automated techniques, and that go beyond simple pixel counting and that apply directly to 3D and 4D analysis. (Length, area, and volume measurements are examples of simple first-order measurements that can be obtained by pixel counting.) Those higher order measures include, but are not limited to:

eigenfunction decompositions

moments of inertia

shape analysis, including local curvature

surface bending energy

shape signatures

results of morphological operations such as skeletonization

fractal analysis

3D wavelet analysis

advanced surface and shape analysis such as a 3D orthogonal basis function with scale invariant properties

trajectories of bones, joints, tendons, and moving musculoskeletal structures.

Mathematical theories of these higher order measurements can be found in Kaye, B. H., “Image Analysis Procedures for Characterizing the Fractal Dimension of Fine Particles,” Proc. Part. Tech. Conference, 1986; Ashton, E. et al., “Spatial-Spectral Anomaly Detection with Shape-Based Classification,” Proc. Military Sensing Symposium on Targets, Backgrounds and Discrimination, 2000; and Struik, D. J., Lectures on Classical Differential Geometry, 2nd ed., New York: Dover, 1988.

The present invention represents a resolution to the needs noted above. Moreover, and in sum, the present invention provides a method and system for the precise and sophisticated measurement of biomarkers, the accurate definition of trends over time, the assessment of biomarkers by measurement of their response to a stimulus and the integration of abnormal biomarker locations with the diagnostic image information.

The measurement of internal organs and structures via medical imaging modalities (i.e., MRI, CT and ultrasound) provides invaluable image data sets for use in a variety of medical fields. These data sets permit medical personnel to objectively measure an object or objects of interest. Such objects may be deemed biomarkers and, per this invention, the inventors choose to define biomarkers as the abnormality and normality of structures, along with their topological, morphological, radiological and pharmacokinetic characteristics and parameters, which may serve as sensitive indicators of disease, disease progress, and any other associated pathological state. For example, a physician examining a cancer patient may employ either MRI or CT scan technology to measure any number of pertinent biomarkers, such as tumor compactness, tumor volume, and/or tumor surface roughness.

The inventors have discovered that the following new biomarkers are sensitive indicators of the progress of diseases characterized by solid tumors in humans and in animals.

The following biomarkers relate to cancer studies. The simplest biomarkers in that category are tumor length, width and 3D volume. Others are:

Tumor surface area

Tumor compactness (surface-to-volume ratio)

Tumor surface curvature

Tumor surface roughness

Necrotic core volume

necrotic core compactness

necrotic core shape

Viable periphery volume

Volume of tumor vasculature

Change in tumor vasculature over time

Tumor shape, as defined through spherical harmonic analysis

Morphological surface characteristics

lesion characteristics

tumor characteristics

tumor peripheral characteristics

tumor core characteristics

bone metastases characteristics

ascites characteristics

pleural fluid characteristics

vessel structure characteristics

neovasculature characteristics

polyp characteristics

nodule characteristics

angiogenisis characteristics

The inventors have also discovered that the following biomarkers are sensitive indicators of osteoarthritis joint disease in humans and in animals:

shape of the subchondral bone plate

layers of the cartilage and their relative size

signal intensity distribution within the cartilage layers

contact area between the articulating cartilage surfaces

surface topology of the cartilage shape

intensity of bone marrow edema

separation distances between bones

meniscus shape

meniscus surface area

meniscus contact area with cartilage

cartilage structural characteristics

cartilage surface characteristics

meniscus structural characteristics

meniscus surface characteristics

pannus structural characteristics

joint fluid characteristics

osteophyte characteristics

bone characteristics

lytic lesion characteristics

prosthesis contact characteristics

prosthesis wear

joint spacing characteristics

tibia medial cartilage volume

Tibia lateral cartilage volume

femur cartilage volume

patella cartilage volume

tibia medial cartilage curvature

tibia lateral cartilage curvature

femur cartilage curvature

patella cartilage curvature

cartilage bending energy

subchondral bone plate curvature

subchondral bone plate bending energy

meniscus volume

osteophyte volume

cartilage T2 lesion volumes

bone marrow edema volume and number

synovial fluid volume

synovial thickening

subchondrial bone cyst volume

kinematic tibial translation

kinematic tibial rotation

kinematic tibial valcus

distance between vertebral bodies

degree of subsidence of cage

degree of lordosis by angle measurement

degree of off-set between vertebral bodies

femoral bone characteristics

patella characteristics

The inventors have also discovered that the following new biomarkers are sensitive indicators of neurological disease in humans and in animals:

The shape, topology, and morphology of brain lesions

The shape, topology, and morphology of brain plaques

The shape, topology, and morphology of brain ischemia

The shape, topology, and morphology of brain tumors

The spatial frequency distribution of the sulci and gyri

The compactness (a measure of surface to volume ratio) of gray matter and white matter

whole brain characteristics

gray matter characteristics

white matter characteristics

cerebral spinal fluid characteristics

hippocampus characteristics

brain sub-structure characteristics

The ratio of cerebral spinal fluid volume to gray mater and white matter volume

The number and volume of brain lesions

The following biomarkers are sensitive indicators of disease and toxicity in organs:

organ volume

organ surface

organ compactness

organ shape

organ surface roughness

fat volume and shape

Once these or similar biomarkers are quantitatively determined, there is a need for combining the biomarker information with the original 2D tomographic images (from MRI, CT, Ultrasound or other tomographic modalities) which are rich in anatomical detail and texture and are typically viewed consecutively on a reader light box or a CRT display. Biomarker parameters, for example the surface roughness of the cartilage of the knee, can be compared with expected values, and locations can be identified where the biomarker parameters are abnormal. These can be color coded on a 3D rendering of the biomarker. In addition, this information can be superimposed or combined with the original radiological image, to highlight the particular region on the 2D tomographic image that corresponds to a voxel in 3D identified by an abnormal biomarker parameter. In this way, a radiologist or surgeon examining the 2D images in the conventional manner can have a computer assisted localization (CAL) that identifies a region of interest that should be examined more closely.

### BRIEF DESCRIPTION OF THE DRAWINGS

A preferred embodiment of the present invention will be set forth in detail with reference to the drawings, in which:

FIG. 1 shows a flow chart of an overview of the process of the preferred embodiment;

FIG. 2 shows a flow chart of a segmentation process used in the process of FIG. 1;

FIG. 3 shows a process of tracking a segmented image in multiple images taken over time;

FIG. 4 shows a block diagram of a system on which the process of FIGS. 1-3 can be implemented;

FIGS. 5a-5e show an example of the present invention in the case of a human knee; and

FIGS. 6a-6e show a further example of the present invention in the case of a human knee.

### DETAILED DESCRIPTION OF THE PREFERRED EMBODIMENT

A preferred embodiment of the present invention will now be set forth with reference to the drawings.

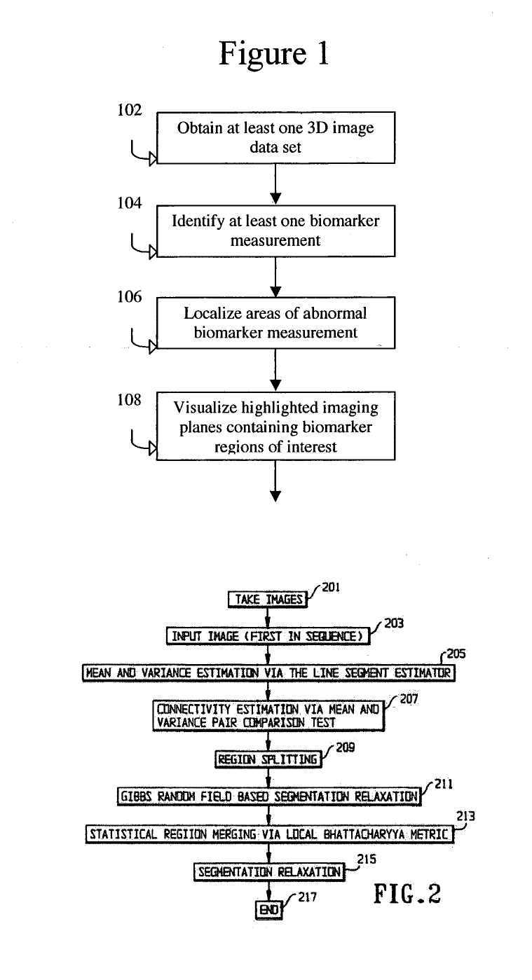

An overview of the operational steps carried out in the preferred embodiment is shown in FIG. 1. In step 102, one or more 3D image data sets are taken in a region of interest in the patient. The 3D image data sets can be taken by any suitable technique, such as MRI; if there are more than one, they are separated by time to form a time sequence of images. In step 104, a biomarker is identified. For example, the biomarkers can be the local roughness, thickness, and curvature of the human knee cartilage. In step 106, biomarker regions of abnormal, extreme, or unexpected values are identified. These particular regions along with the normal or expected values are defined by reference to data, including norms or expected values for that patient. These can be derived in a number of ways: from a-priori data on other patients of similar condition; from the current patient's 3D biomarker parameters and their extrema, or from a 4D model representing the change over time of the biomarker. In step 108, the original scan planes and their intersections with the regions of abnormal biomarker parameters are identified and highlighted. In this way, the radiologist can view the 2D images in the conventional manner, but with extra attention to those localized regions that are highlighted due to the biomarker analysis.

The extraction of the biomarker information in step 104 will now be explained. Conventionally, structures of interest have been identified by experts, such as radiologists, with manual tracing. As previously mentioned, the manual and semi-manual tracings of images lead to high intra- and inter-observer variability. The preferred method for extracting the biomarkers is with statistical based reasoning as defined in Parker et al (U.S. Pat. No. 6,169,817), whose disclosure is hereby incorporated by reference in its entirety into the present disclosure. From raw image data obtained through magnetic resonance imaging or the like, an object is reconstructed and visualized in four dimensions (both space and time) by first dividing the first image in the sequence of images into regions through statistical estimation of the mean value and variance of the image data and joining of picture elements (voxels) that are sufficiently similar and then extrapolating the regions to the remainder of the images by using known motion characteristics of components of the image (e.g., spring constants of muscles and tendons) to estimate the rigid and deformational motion of each region from image to image. The object and its regions can be rendered and interacted with in a four-dimensional (4D) virtual reality environment, the four dimensions being three spatial dimensions and time.

The segmentation will be explained with reference to FIG. 2. First, at step 201, the images in the sequence are taken, as by an MRI. Raw image data are thus obtained. Then, at step 203, the raw data of the first image in the sequence are input into a computing device. Next, for each voxel, the local mean value and region variance of the image data are estimated at step 205. The connectivity among the voxels is estimated at step 207 by a comparison of the mean values and variances estimated at step 205 to form regions. Once the connectivity is estimated, it is determined which regions need to be split, and those regions are split, at step 209. The accuracy of those regions can be improved still more through the segmentation relaxation of step 211. Then, it is determined which regions need to be merged, and those regions are merged, at step 213. Again, segmentation relaxation is performed, at step 215. Thus, the raw image data are converted into a segmented image, which is the end result at step 217. Further details of any of those processes can be found in the above-cited Parker et al patent.

The creation of a 4D model (in three dimensions of space and one of time) will be described with reference to FIG. 3. A motion tracking and estimation algorithm provides the information needed to pass the segmented image from one frame to another once the first image in the sequence and the completely segmented image derived therefrom as described above have been input at step 301. The presence of both the rigid and non-rigid components should ideally be taken into account in the estimation of the 3D motion. According to the present invention, the motion vector of each voxel is estimated after the registration of selected feature points in the image.

To take into consideration the movement of the many structures present in a joint, the approach of the present invention takes into account the local deformations of soft tissues by using a priori knowledge of the material properties of the different structures found in the image segmentation. Such knowledge is input in an appropriate database form at step 303. Also, different strategies can be applied to the motion of the rigid structures and to that of the soft tissues. Once the selected points have been registered, the motion vector of every voxel in the image is computed by interpolating the motion vectors of the selected points. Once the motion vector of each voxel has been estimated, the segmentation of the next image in the sequence is just the propagation of the segmentation of the former image. That technique is repeated until every image in the sequence has been analyzed.

The definition of time and the order of a sequence can be reversed for the purpose of the analysis. For example, in a time series of cancer lesions in the liver, there may be more lesions in the final scan than were present in the initial scan. Thus, the 4D model can be run in the reverse direction to make sure all lesions are accounted for. Similarly, a long time series can be run from a mid-point, with analysis proceeding both forward and backward from the mid-point.

Finite-element models (FEM) are known for the analysis of images and for time-evolution analysis. The present invention follows a similar approach and recovers the point correspondence by minimizing the total energy of a mesh of masses and springs that models the physical properties of the anatomy. In the present invention, the mesh is not constrained by a single structure in the image, but instead is free to model the whole volumetric image, in which topological properties are supplied by the first segmented image and the physical properties are supplied by the a priori properties and the first segmented image. The motion estimation approach is an FEM-based point correspondence recovery algorithm between two consecutive images in the sequence. Each node in the mesh is an automatically selected feature point of the image sought to be tracked, and the spring stiffness is computed from the first segmented image and a priori knowledge of the human anatomy and typical biomechanical properties for muscle, bone and the like.

Many deformable models assume that a vector force field that drives spring-attached point masses can be extracted from the image. Most such models use that approach to build semi-automatic feature extraction algorithms. The present invention employs a similar approach and assumes that the image sampled at t=n is a set of three dynamic scalar fields:

Φ(x,t)={gₙ(x),|∇gₙ(x)|,∇²gₙ(x)},

namely, the gray-scale image value, the magnitude of the gradient of the image value, and the Laplacian of the image value. Accordingly, a change in Φ(x, t) causes a quadratic change in the scalar field energy U_{Φ}(x)∝(ΔΦ(x))². Furthermore, the structures underlying the image are assumed to be modeled as a mesh of spring-attached point masses in a state of equilibrium with those scalar fields. Although equilibrium assumes that there is an external force field, the shape of the force field is not important. The distribution of the point masses is assumed to change in time, and the total energy change in a time period Δt after time t=n is given by

$$\begin{matrix}Δ {U}_{n} ( Δ x ) = {\sum }_{∀ X ∈ {g}_{n}}^{} [ {( α ( {g}_{n} ( x ) - {g}_{n + 1} ( x + Δ x ) ) )}^{2} + \\ {( β ( \vert ∇ {g}_{n} ( x ) \vert - \vert ∇ {g}_{n + 1} ( x + Δ x ) \vert ) )}^{2} + \\ {( γ ( {∇}^{2} {g}_{n} ( x ) + {∇}^{2} {g}_{n + 1} ( x + Δ x ) ) )}^{2} + \frac{1}{2} η Δ {X}^{T} K Δ X ]\end{matrix}$$

where α, β, and γ are weights for the contribution of every individual field change, 11 weighs the gain in the strain energy, K is the FEM stiffness matrix, and ΔX is the FEM node displacement matrix. Analysis of that equation shows that any change in the image fields or in the mesh point distribution increases the system total energy. Therefore, the point correspondence from gₙ to gₙ₊₁ is given by the mesh configuration whose total energy variation is a minimum. Accordingly, the point correspondence is given by

\hat{X}=X+Δ\hat{X}

where

Δ\hat{X}=min_{ΔX}ΔUₙ(ΔX).

In that notation, mine q is the value of p that minimizes q.

While the equations set forth above could conceivably be used to estimate the motion (point correspondence) of every voxel in the image, the number of voxels, which is typically over one million, and the complex nature of the equations make global minimization difficult. To simplify the problem, a coarse FEM mesh is constructed with selected points from the image at step 305. The energy minimization gives the point correspondence of the selected points.

The selection of such points is not trivial. First, for practical purposes, the number of points has to be very small, typically ≅10⁴; care must be taken that the selected points describe the whole image motion. Second, region boundaries are important features because boundary tracking is enough for accurate region motion description. Third, at region boundaries, the magnitude of the gradient is high, and the Laplacian is at a zero crossing point, making region boundaries easy features to track. Accordingly, segmented boundary points are selected in the construction of the FEM.

Although the boundary points represent a small subset of the image points, there are still too many boundary points for practical purposes. In order to reduce the number of points, constrained random sampling of the boundary points is used for the point extraction step. The constraint consists of avoiding the selection of a point too close to the points already selected. That constraint allows a more uniform selection of the points across the boundaries. Finally, to reduce the motion estimation error at points internal to each region, a few more points of the image are randomly selected using the same distance constraint. Experimental results show that between 5,000 and 10,000 points are enough to estimate and describe the motion of a typical volumetric image of 256×256×34 voxels. Of the selected points, 75% are arbitrarily chosen as boundary points, while the remaining 25% are interior points. Of course, other percentages can be used where appropriate.

Once a set of points to track is selected, the next step is to construct an FEM mesh for those points at step 307. The mesh constrains the kind of motion allowed by coding the material properties and the interaction properties for each region. The first step is to find, for every nodal point, the neighboring nodal point. Those skilled in the art will appreciate that the operation of finding the neighboring nodal point corresponds to building the Voronoi diagram of the mesh. Its dual, the Delaunay triangulation, represents the best possible tetrahedral finite element for a given nodal configuration. The Voronoi diagram is constructed by a dilation approach. Under that approach, each nodal point in the discrete volume is dilated. Such dilation achieves two purposes. First, it is tested when one dilated point contacts another, so that neighboring points can be identified. Second, every voxel can be associated with a point of the mesh.

Once every point xᵢ has been associated with a neighboring point xⱼ, the two points are considered to be attached by a spring having spring constant k_{i,j}^{l,m}, where l and m identify the materials. The spring constant is defined by the material interaction properties of the connected points; those material interaction properties are predefined by the user in accordance with known properties of the materials. If the connected points belong to the same region, the spring constant reduces to k_{i,j}^{l,m} and is derived from the elastic properties of the material in the region. If the connected points belong to different regions, the spring constant is derived from the average interaction force between the materials at the boundary. If the object being imaged is a human shoulder, the spring constant can be derived from a table such as the following:

| | Humeral head | Muscle | Tendon | Cartilage |
|---|---|---|---|---|
| Humeral head | 10⁴ | 0.15 | 0.7 | 0.01 |
| Muscle | 0.15 | 0.1 | 0.7 | 0.6 |
| Tendon | 0.7 | 0.7 | 10 | 0.01 |
| Cartilage | 0.01 | 0.6 | 0.01 | 10² |

In theory, the interaction must be defined between any two adjacent regions. In practice, however, it is an acceptable approximation to define the interaction only between major anatomical components in the image and to leave the rest as arbitrary constants. In such an approximation, the error introduced is not significant compared with other errors introduced in the assumptions set forth above.

Spring constants can be assigned automatically, as the approximate size and image intensity for the bones are usually known a priori. Segmented image regions matching the a priori expectations are assigned to the relatively rigid elastic constants for bone. Soft tissues and growing or shrinking lesions are assigned relatively soft elastic constants.

Once the mesh has been set up, the next image in the sequence is input at step 309, and the energy between the two successive images in the sequence is minimized at step 311. The problem of minimizing the energy U can be split into two separate problems: minimizing the energy associated with rigid motion and minimizing that associated with deformable motion. While both energies use the same energy function, they rely on different strategies.

The rigid motion estimation relies on the fact that the contribution of rigid motion to the mesh deformation energy (ΔX^{T}KΔX)/2 is very close to zero. The segmentation and the a priori knowledge of the anatomy indicate which points belong to a rigid body. If such points are selected for every individual rigid region, the rigid motion energy minimization is accomplished by finding, for each rigid region Rᵢ, the rigid motion rotation Rᵢ and the translation Tᵢ that minimize that region's own energy:

$$Δ {X}_{rigid} = {min}_{Δ x} {U}_{rigid} = {\sum }_{∀ l ∈ rigid}^{} ( Δ \hat{X} = {min}_{Δ {x}_{i}} {U}_{n} ( Δ {X}_{i} ) )$$

where ΔXᵢ=Rᵢ−Xᵢ+TᵢXᵢ and Δ\hat{x}ᵢ is the optimum displacement matrix for the points that belong to the rigid region Rᵢ. That minimization problem has only six degrees of freedom for each rigid region: three in the rotation matrix and three in the translation matrix. Therefore, the twelve components (nine rotational and three translational) can be found via a six-dimensional steepest-descent technique if the difference between any two images in the sequence is small enough.

Once the rigid motion parameters have been found, the deformational motion is estimated through minimization of the total system energy U. That minimization cannot be simplified as much as the minimization of the rigid energy, and without further considerations, the number of degrees of freedom in a 3D deformable object is three times the number of node points in the entire mesh. The nature of the problem allows the use of a simple gradient descent technique for each node in the mesh. From the potential and kinetic energies, the Lagrangian (or kinetic potential, defined in physics as the kinetic energy minus the potential energy) of the system can be used to derive the Euler-Lagrange equations for every node of the system where the driving local force is just the gradient of the energy field. For every node in the mesh, the local energy is given by

$$\begin{matrix}{U}_{{x}_{i} n} ( Δ x ) = {( α ( {g}_{n} ( {x}_{i} + Δ x ) - {g}_{n + 1} ( {x}_{i} ) ) )}^{2} + \\ {( β ( \vert ∇ {g}_{n} ( {x}_{i} + Δ x ) \vert + \vert ∇ {g}_{n + 1} ( {x}_{i} ) \vert ) )}^{2} + \\ {γ ( {∇}^{2} {g}_{n} ( {x}_{i} + Δ x ) + {∇}^{2} {g}_{n + 1} ( {x}_{i} ) )}^{2} + \\ \frac{1}{2} η {\sum }_{{x}_{i} ∈ {G}_{m} ( {X}_{i} )}^{} {( {k}_{i , j}^{l , m} ( {x}_{j} - {x}_{i} - Δ x ) )}^{2}\end{matrix}$$

where Gₘ represents a neighborhood in the Voronoi diagram.

Thus, for every node, there is a problem in three degrees of freedom whose minimization is performed using a simple gradient descent technique that iteratively reduces the local node energy. The local node gradient descent equation is

xᵢ(n+1)=xᵢ(n)−vΔU₍ₓ_{ᵢ}_{(n),n)}(Δx)

where the gradient of the mesh energy is analytically computable, the gradient of the field energy is numerically estimated from the image at two different resolutions, x(n+1) is the next node position, and v is a weighting factor for the gradient contribution.

At every step in the minimization, the process for each node takes into account the neighboring nodes' former displacement. The process is repeated until the total energy reaches a local minimum, which for small deformations is close to or equal to the global minimum. The displacement vector thus found represents the estimated motion at the node points.

Once the minimization process just described yields the sampled displacement field ΔX, that displacement field is used to estimate the dense motion field needed to track the segmentation from one image in the sequence to the next (step 313). The dense motion is estimated by weighting the contribution of every neighbor mode in the mesh. A constant velocity model is assumed, and the estimated velocity of a voxel x at a time t is v(x, t)=Δx(t)/Δt. The dense motion field is estimated by

$$v ( x , t ) = \frac{c ( x )}{Δ t} {\sum }_{∀ Δ {x}_{j} ∈ {G}_{m} ( {x}_{i} )}^{} \frac{{k}^{l , m} Δ {x}_{j}}{\vert x - {x}_{j} \vert}$$

$$where$$

$$c ( x ) = {[ {\sum }_{∀ Δ {x}_{j} ∈ {G}_{m} ( {x}_{i} )}^{} \frac{{k}^{l , m}}{\vert x - {x}_{j} \vert} ]}^{- 1}$$

k^{l,m} is the spring constant or stiffness between the materials l and m associated with the voxels x and xⱼ, Δt is the time interval between successive images in the sequence, |x−xⱼ| is the simple Euclidean distance between the voxels, and the interpolation is performed using the neighbor nodes of the closest node to the voxel x. That interpolation weights the contribution of every neighbor node by its material property k_{i,j}^{l,m}; thus, the estimated voxel motion is similar for every homogeneous region, even at the boundary of that region.

Then, at step 315, the next image in the sequence is filled with the segmentation data. That means that the regions determined in one image are carried over into the next image. To do so, the velocity is estimated for every voxel in that next image. That is accomplished by a reverse mapping of the estimated motion, which is given by

$$v ( x , t + Δ t ) = \frac{1}{H} {\sum }_{∀ [ {x}_{j} + v ( {x}_{j} , t ) ] ∈ S ( x )}^{} v ( {x}_{j} , t )$$

where H is the number of points that fall into the same voxel space S(x) in the next image. That mapping does not fill all the space at time t+Δt, but a simple interpolation between mapped neighbor voxels can be used to fill out that space. Once the velocity is estimated for every voxel in the next image, the segmentation of that image is simply

L(x,t+Δt)=L(x−v(x,t+Δt)Δt,t)

where L(x,t) and L(x,t+Δt) are the segmentation labels at the voxel x for the times t and t+At.

At step 317, the segmentation thus developed is adjusted through relaxation labeling, such as that done at steps 211 and 215, and fine adjustments are made to the mesh nodes in the image. Then, the next image is input at step 309, unless it is determined at step 319 that the last image in the sequence has been segmented, in which case the operation ends at step 321.

First-order measurements—length, diameter, and their extensions to area and volume—are quite useful quantities. However, they are limited in their ability to assess subtle but potentially important features of tissue structures or substructures. Thus, the inventors propose to use higher-order measurements of structure and shape to characterize biomarkers. The inventors define higher-order measures as any measurements that cannot be extracted directly from the data using traditional manual or semi-automated techniques and that go beyond simple pixel counting. Examples are given above.

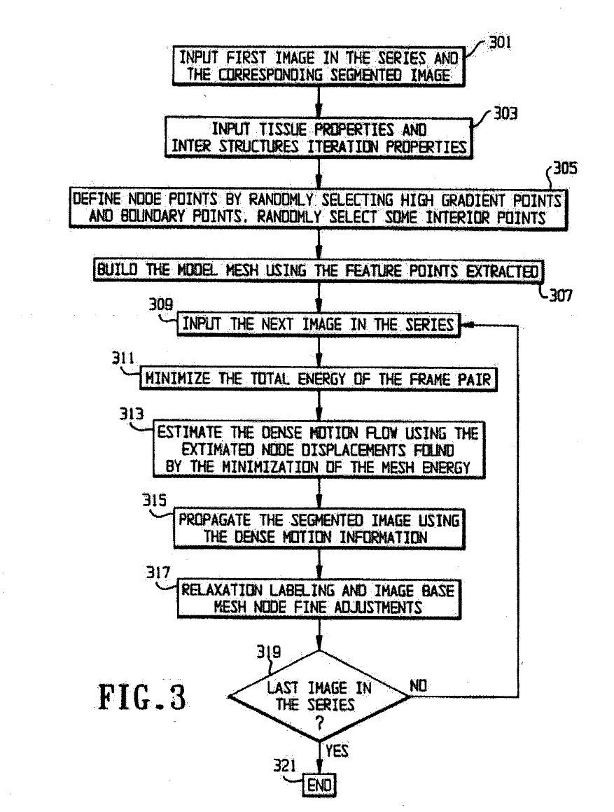

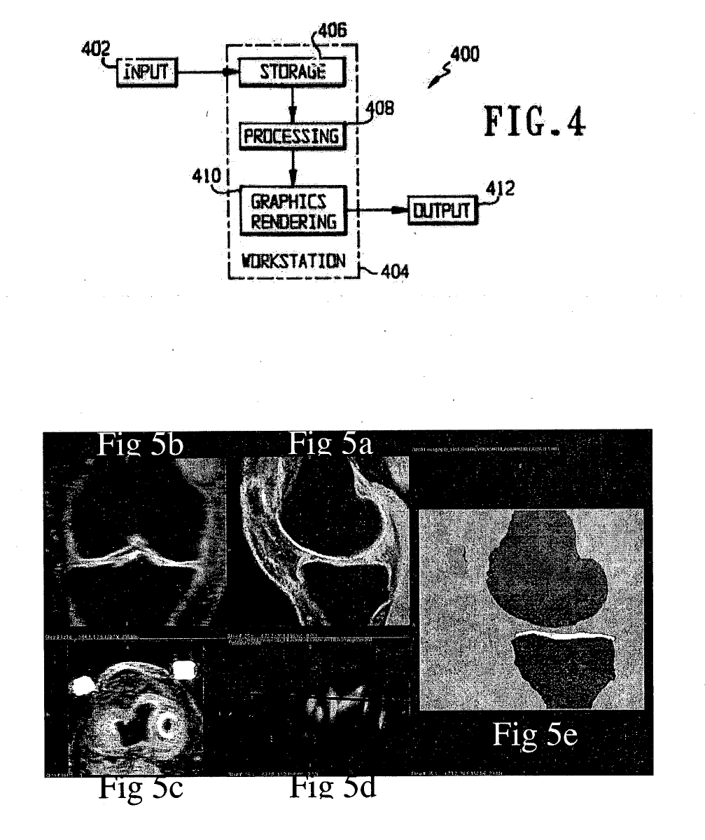

The operations described above can be implemented in a system such as that shown in the block diagram of FIG. 4. System 400 includes an input device 402 for input of the image data, the database of material properties, and the like. The information input through the input device 402 is received in the workstation 404, which has a storage device 406 such as a hard drive, a processing unit 408 for performing the processing disclosed above to provide the 4D data, and a graphics rendering engine 410 for preparing the 4D data for viewing, e.g., by surface rendering. An output device 412 can include a monitor for viewing the images rendered by the rendering engine 410, a further storage device such as a video recorder for recording the images, or both. Illustrative examples of the workstation 304 and the graphics rendering engine 410 are a Silicon Graphics Indigo workstation and an Irix Explorer 3D graphics engine.

Shape and topology of the identified biomarkers can be quantified by any suitable techniques known in analytical geometry. The preferred method for quantifying shape and topology is with the morphological and topological formulas as defined by the following references:

Shape Analysis and Classification, L. Costa and R. Cesar, Jr., CRC Press, 2001.

Curvature Analysis: Peet, F. G., Sahota, T. S. “Surface Curvature as a Measure of Image Texture” IEEE Transactions on Pattern Analysis and Machine Intelligence 1985 Vol PAMI-7 G:734-738.

Struik, D. J., Lectures on Classical Differential Geometry, 2nd ed., Dover, 1988.

Shape and Topological Descriptors: Duda, R. O, Hart, P. E., Pattern Classification and Scene Analysis, Wiley & Sons, 1973.

Jain, A. K, “Fundamentals of Digital Image Processing,” Prentice Hall, 1989.

Spherical Harmonics: Matheny, A., Goldgof, D. “The Use of Three and Four Dimensional Surface Harmonics for Nonrigid Shape Recovery and Representation,” IEEE Transactions on Pattern Analysis and Machine Intelligence 1995, 17: 967-981; Chen, C. W, Huang, T. S., Arrot, M. “Modeling, Analysis, and Visualization of Left Ventricle Shape and Motion by Hierarchical Decomposition,” IEEE Transactions on Pattern Analysis and Machine Intelligence 1994, 342-356.

Those morphological and topological measurements have not in the past been applied to biomarkers which have a progressive, non-periodic change over time.

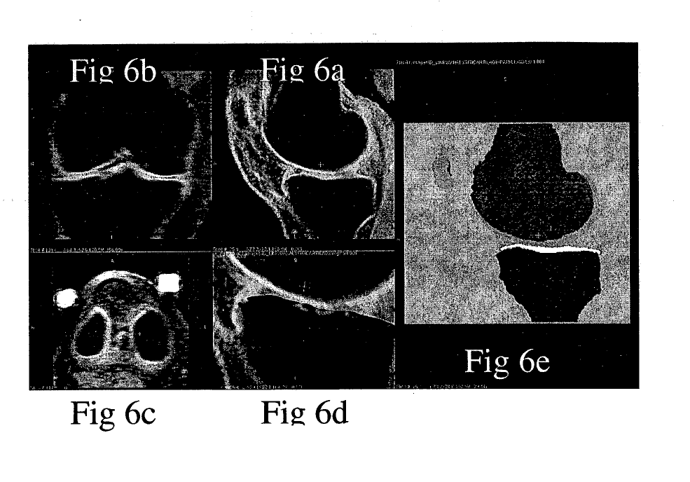

Illustrative examples of the invention will be set forth with reference to FIGS. 5a-5e and 6a-6e. FIG. 5a demonstrates a conventional MRI sagittal view of a human knee. The cartilage is a thin layer that is difficult to discriminate in a single 2D scan. FIGS. 5b and 5c demonstrate conventional reformatting and display of the 3D data set, showing coronal and transverse planes, respectively. The cartilage is particularly difficult to assess in the conventional transverse plane, FIG. 5c, since the cartilage is not conveniently shaped flat so it will not fall into a single transverse plane. However, using the advanced segmentation methods described in this invention, the complete cartilage layers from both the femur and the tibia can be separated and identified. These are shown as individual layers in FIG. 5e, which is a sagittal view similar to that of FIG. 5a, however demonstrating the segmented and identified bone and cartilage structures. A separate coronal view of the entire tibial cartilage is given in FIG. 5d, as a surface rendering with shading (colors can also be used) indicating the local curvature of the cartilage surface based on a 3D analysis of the entire cartilage. Although this is a coronal view, it is not a single slice but rather demonstrates, in a surface rendering, the entire tibial cartilage surface along with the measured parameters of local surface curvature indicated in different shades. Some extreme values of negative (concave) curvature are indicated as very light regions. The black stripe indicates the location of the sagittal plane shown simultaneously in FIG. 5a.

FIG. 6a demonstrates again the sagittal view of a human knee, and FIGS. 6b and 6c demonstrate corresponding coronal and transverse views of the same volumetric MRI data. FIG. 6e demonstrates the segmented and identified femur, tibia, and their associated cartilage layers. FIG. 6d illustrates the superposition of the cartilage local curvature measurements, obtained from a 3D analysis of the segmented tibial cartilage layer, with a zoom view of the sagittal image slice of the knee conventionally examined by the radiologist or other imaging expert. In this way, quantitative information derived from 3D or 4D biomarker measurements can be visualized along with the conventional 2D tomographic image that is conventionally reviewed by imaging experts. Locations of extreme local curvature are very difficult to identify on any single 2D sagittal slice, but these locations are quickly identified in the combined view, FIG. 6d, with the use of color overlay in this example to encode curvature. Other biomarkers can be similarly analyzed and a number of means of highlighting, including the use of color, of blinking regions, or arrows, can similarly be employed to identify the location of extreme or abnormal biomarker parameters.

While a preferred embodiment of the invention has been set forth above, those skilled in the art who have reviewed the present disclosure will readily appreciate that other embodiments can be realized within the scope of the present invention. For example, any suitable imaging technology can be used. Therefore, the present invention should be construed as limited only by the appended claims.

We claim:

1. A method for assessing a region of interest in a patient, the method comprising:
(a) taking at least one three-dimensional image of the region of interest;
(b) identifying at least one biomarker in the at least one three-dimensional image; and
(c) determining whether the at least one biomarker identified in step (b) is characterized by an abnormal biomarker parameter.

2. The method of claim 1, wherein the at least one three-dimensional image comprises a plurality of three-dimensional images taken over time.

3. The method of claim 2, wherein the at least one biomarker comprises a four-dimensional biomarker having three spatial dimensions and one time dimension.

4. The method of claim 1, wherein step (c) comprises:
(i) determining a biomarker parameter which characterizes the at least one biomarker;
(ii) comparing the biomarker parameter determined in step (c)(i) with a range of normal biomarker parameters;
(iii) if the biomarker parameter is within the range of normal biomarker parameters, determining that the biomarker is not characterized by the abnormal biomarker parameter; and
(iv) if the biomarker parameter is not within the range of normal biomarker parameters, determining that the biomarker is characterized by the abnormal biomarker parameter.

5. The method of claim 1, wherein step (c) is performed voxel by voxel for each of a plurality of voxels corresponding to the at least one biomarker.

6. The method of claim 1, wherein, if it is determined in step (c) that the at least one biomarker is characterized by an abnormal biomarker parameter, the method further comprises (d) providing a visual representation of the at least one biomarker.

7. The method of claim 6, wherein step (d) comprises highlighting a location of the at least one biomarker having the abnormal biomarker parameter on an image of the region of interest.

8. The method of claim 7, wherein the image on which the location is highlighted is a two-dimensional image.

9. The method of claim 8, wherein the two-dimensional image is a radiological image.

10. The method of claim 1, wherein the at least one biomarker comprises a cancer-related biomarker.

11. The method of claim 10, wherein the cancer-related biomarker comprises a biomarker selected from the group consisting of:
tumor surface area;
tumor compactness;
tumor surface curvature;
tumor surface roughness;
necrotic core volume;
necrotic core compactness;
necrotic core shape;
viable periphery volume;
volume of tumor vasculature;
change in tumor vasculature over time;
tumor shape;
morphological surface characteristics;
lesion characteristics;
tumor characteristics;
tumor peripheral characteristics;
tumor core characteristics;
bone metastases characteristics;
ascites characteristics;
pleural fluid characteristics;
vessel structure characteristics;
neovasculature characteristics;
polyp characteristics;
nodule characteristics; and
angiogenisis characteristics.

12. The method of claim 10, wherein the cancer-related biomarker comprises a biomarker selected from the group consisting of:
tumor length;
tumor width; and
tumor 3d volume.

13. The method of claim 1, wherein the at least one biomarker comprises a joint-related biomarker.

14. The method of claim 13, wherein the joint-related biomarker is selected from the group consisting of:
shape of a subchondral bone plate;
layers of cartilage and their relative size;
signal intensity distribution within cartilage layers;
contact area between articulating cartilage surfaces;
surface topology of cartilage shape;
intensity of bone marrow edema;
separation distances between bones;
meniscus shape;
meniscus surface area;
meniscus contact area with cartilage;
cartilage structural characteristics;
cartilage surface characteristics;
meniscus structural characteristics;
meniscus surface characteristics;
pannus structural characteristics;
joint fluid characteristics;
osteophyte characteristics;
bone characteristics;
lytic lesion characteristics;
prosthesis contact characteristics;
prosthesis wear;
joint spacing characteristics;
tibia medial cartilage volume;
tibia lateral cartilage volume;
femur cartilage volume;
patella cartilage volume;
tibia medial cartilage curvature;
tibia lateral cartilage curvature;
femur cartilage curvature;
patella cartilage curvature;
cartilage bending energy;
subchondral bone plate curvature;
subchondral bone plate bending energy;
meniscus volume;
osteophyte volume;
cartilage t2 lesion volumes;
bone marrow edema volume and number;
synovial fluid volume;
synovial thickening;
subchondrial bone cyst volume and number;
kinematic tibial translation;
kinematic tibial rotation;
kinematic tibial valcus;
distance between vertebral bodies;
degree of subsidence of cage;
degree of lordosis by angle measurement;
degree of off-set between vertebral bodies;
femoral bone characteristics; and
patella characteristics.

15. The method of claim 1, wherein the at least one biomarker comprises a neurological biomarker.

16. The method of claim 15, wherein the neurological biomarker is selected from the group consisting of:
a shape, topology, and morphology of brain lesions;
a shape, topology, and morphology of brain plaques;
a shape, topology, and morphology of brain ischemia;
a shape, topology, and morphology of brain tumors;
a spatial frequency distribution of the sulci and gyri;
compactness of gray matter and white matter;
whole brain characteristics;
gray matter characteristics;
white matter characteristics;
cerebral spinal fluid characteristics;
hippocampus characteristics;
brain sub-structure characteristics;
a ratio of cerebral spinal fluid volume to gray matter and white matter volume; and
a number and volume of brain lesions.

17. The method of claim 1, wherein the region of interest comprises an organ of the patient, and wherein the at least one biomarker comprises a biomarker relating to disease or toxicity in the organ.

18. The method of claim 17, wherein the biomarker relating to disease or toxicity in the organ is selected from the group consisting of:
organ volume;
organ surface;
organ compactness;
organ shape;
organ surface roughness; and
fat volume and shape.

19. The method of claim 1, wherein the at least one biomarker comprises a higher-order measure.

20. The method of claim 19, wherein the higher-order measure is selected from the group consisting of:
eigenfunction decompositions;
moments of inertia;
shape analysis, including local curvature;
results of morphological operations such as skeletonization;
fractal analysis; 3D wavelet analysis;
advanced surface and shape analysis such as a 3D spherical harmonic analysis with scale invariant properties; and
trajectories of bones, joints, tendons, and moving musculoskeletal structures.

21. A system for assessing a region of interest of a patient, the system comprising:
(a) an input device for receiving at least one three-dimensional image of the region of interest;
(b) a processor, in communication with the input device, for receiving the at least one three-dimensional image of the region of interest from the input device, for identifying at least one biomarker in the at least one three-dimensional image, and for determining whether the at least one biomarker is characterized by an abnormal biomarker parameter; and
(c) an output device for displaying the at least one three-dimensional image, the identification of the at least one biomarker and an indication of whether the at least one biomarker is characterized by the abnormal biomarker parameter.

22. The system of claim 21, wherein the at least one three-dimensional image comprises a plurality of three-dimensional images taken over time.

23. The system of claim 22, wherein the at least one biomarker comprises a four-dimensional biomarker having three spatial dimensions and one time dimension.

24. The system of claim 21, wherein the processor determines whether the at least one biomarker is characterized by the abnormal biomarker parameter by:
(i) determining a biomarker parameter which characterizes the at least one biomarker;
(ii) comparing the biomarker parameter determined in step (i) with a range of normal biomarker parameters;
(iii) if the biomarker parameter is within the range of normal biomarker parameters, determining that the biomarker is not characterized by the abnormal biomarker parameter; and
(iv) if the biomarker parameter is not within the range of normal biomarker parameters, determining that the biomarker is characterized by the abnormal biomarker parameter.

25. The system of claim 21, wherein the processor determines whether the at least one biomarker is characterized by the abnormal biomarker parameter by performing a determination voxel by voxel for each of a plurality of voxels corresponding to the at least one biomarker.

26. The system of claim 21, wherein, if the processor determines that the at least one biomarker is characterized by the abnormal biomarker parameter, the indication displayed on the output comprises a visual representation of the at least one biomarker.

27. The system of claim 26, wherein the output highlights a location of the at least one biomarker having the abnormal biomarker parameter on an image of the region of interest.

28. The system of claim 27, wherein the image on which the location is highlighted is a two-dimensional image.

29. The system of claim 28, wherein the two-dimensional image is a radiological image.

30. The system of claim 21, wherein the at least one biomarker comprises a cancer-related biomarker.

31. The system of claim 30, wherein the cancer-related biomarker comprises a biomarker selected from the group consisting of:
tumor surface area;
tumor compactness;
tumor surface curvature;
tumor surface roughness;
necrotic core volume;
necrotic core compactness;
necrotic core shape;
viable periphery volume;
volume of tumor vasculature;
change in tumor vasculature over time;
tumor shape;
morphological surface characteristics;
lesion characteristics;
tumor characteristics;
tumor peripheral characteristics;
tumor core characteristics;
bone metastases characteristics;
ascites characteristics;
pleural fluid characteristics;
vessel structure characteristics;
neovasculature characteristics;
polyp characteristics;
nodule characteristics; and
angiogenisis characteristics.

32. The system of claim 30, wherein the cancer-related biomarker comprises a biomarker selected from the group consisting of:
tumor length;
tumor width; and
tumor 3d volume.

33. The system of claim 21, wherein the at least one biomarker comprises a joint-related biomarker.

34. The system of claim 33, wherein the joint-related biomarker is selected from the group consisting of:
shape of a subchondral bone plate;
layers of cartilage and their relative size;
signal intensity distribution within cartilage layers;
contact area between articulating cartilage surfaces;
surface topology of cartilage shape;
intensity of bone marrow edema;
separation distances between bones;
meniscus shape;
meniscus surface area;
meniscus contact area with cartilage;
cartilage structural characteristics;
cartilage surface characteristics;
meniscus structural characteristics;
meniscus surface characteristics;
pannus structural characteristics;
joint fluid characteristics;
osteophyte characteristics;
bone characteristics;
lytic lesion characteristics;
prosthesis contact characteristics;
prosthesis wear;
joint spacing characteristics;
tibia medial cartilage volume;
tibia lateral cartilage volume;
femur cartilage volume;
patella cartilage volume;
tibia medial cartilage curvature;
tibia lateral cartilage curvature;
femur cartilage curvature;
patella cartilage curvature;
cartilage bending energy;
subchondral bone plate curvature;
subchondral bone plate bending energy;
meniscus volume;
osteophyte volume;
cartilage t2 lesion volumes;
bone marrow edema volume and number;
synovial fluid volume;
synovial thickening;
subchondrial bone cyst volume and number;
kinematic tibial translation;
kinematic tibial rotation;
kinematic tibial valcus;
distance between vertebral bodies;
degree of subsidence of cage;
degree of lordosis by angle measurement;
degree of off-set between vertebral bodies;
femoral bone characteristics; and
patella characteristics.

35. The system of claim 21, wherein the at least one biomarker comprises a neurological biomarker.

36. The system of claim 35, wherein the neurological biomarker is selected from the group consisting of:
a shape, topology, and morphology of brain lesions;
a shape, topology, and morphology of brain plaques;
a shape, topology, and morphology of brain ischemia;
a shape, topology, and morphology of brain tumors;
a spatial frequency distribution of the sulci and gyri;
compactness of gray matter and white matter;
whole brain characteristics;
gray matter characteristics;
white matter characteristics;
cerebral spinal fluid characteristics;
hippocampus characteristics;
brain sub-structure characteristics;
a ratio of cerebral spinal fluid volume to gray matter and white matter volume; and
a number and volume of brain lesions.

37. The system of claim 21, wherein the region of interest comprises an organ of the patient, and wherein the at least one biomarker comprises a biomarker relating to disease or toxicity in the organ.

38. The system of claim 37, wherein the biomarker relating to disease or toxicity in the organ is selected from the group consisting of:
organ volume;
organ surface;
organ compactness;
organ shape;
organ surface roughness; and
fat volume and shape.

39. The system of claim 21, wherein the at least one biomarker comprises a higher-order measure.

40. The system of claim 39, wherein the higher-order measure is selected from the group consisting of:
eigenfunction decompositions;
moments of inertia;
shape analysis, including local curvature;
results of morphological operations such as skeletonization;
fractal analysis;
3D wavelet analysis;
advanced surface and shape analysis such as a 3D spherical harmonic analysis with scale invariant properties; and
trajectories of bones, joints, tendons, and moving musculoskeletal structures.
